(19) United States
(12) Patent Application Publication
(10) Pub. No.: US 2004/0198833 A1
(43) Pub. Date: Oct. 7, 2004

(54) Acetylenic sulfonamide derivatives

(76) Inventors: David Michael Bender, indianapolis, IN (US); Scott Louis Forman, Indianapolis, IN (US); Winton Dennis Jones, Fishers, IN (US); Daryl Lynn Smith, Fishers, IN (US); Dennis Michael Zimmermann, Zionsville, IN (US)

Correspondence Address: ELI LILLY AND COMPANY, PATENT DIVISION, P.O. BOX 6288, INDIANAPOLIS, IN, 46206-6288, US

(21) Appl. No.: 10/343,732

(22) PCT Filed: Aug. 10, 2001
(86) PCT No.: PCT/US01/22592

Related U.S. Application Data
(60) Provisional application No. 60/229,394, filed on Aug. 31, 2000.

(51) Int. Cl. A61K 31/18; A61K 31/24; C 07C 3 9/41

(52) U.S. Cl. 514/602; 514/540; 564/86

(57) ABSTRACT

The present invention provides compounds of formula (I), useful for potentiating glutamate receptor function in a mammal and therefore, useful for treating a wide variety of conditions, such as psychiatric and neurological disorders.

In the mammalian central nervous system (CNS), the transmission of nerve impulses is controlled by the interaction between a neurotransmitter, that is released by a sending neuron, and a surface receptor on a receiving neuron, which causes excitation of this receiving neuron. L-Glutamate, which is the most abundant neurotransmitter in the CNS, mediates the major excitatory pathway in mammals, and is referred to as an excitatory amino acid (EM). The receptors that respond to glutamate are called excitatory amino acid receptors (EM receptors). See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term is potentiation (learning and memory), the development of synaptic plasticity, motor control, respiration, cardiovascular regulation, and sensory perception.

Excitatory amino acid receptors are classified into two general types. Receptors that are directly coupled to the opening of cation channels in the cell membrane of the neurons are termed “ionotropic”. This type of receptor has been subdivided into at least three subtypes, which are defined by the depolarizing actions of the selective agonists N-methyl-D-aspartate (NMDA), alpha-amino-3-hydroxy-5-methylisoxazole-4-propionic acid (AMPA), and kainic acid (KA). The second general type of receptor is the G-protein or second messenger-linked “metabotropic” excitatory amino acid receptor. This second type is coupled to multiple second messenger systems that lead to enhanced phosphoinositide hydrolysis, activation of phospholipase D, increases or decreases in c-AMP formation, and changes in ion channel function. Schoepp and Conn, Trends in Pharmacol. Sci., 14, 13 (1993). Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

AMPA receptors are assembled from four protein sub-units known as GluR1 to GluR4, while kainic acid receptors are assembled from the sub-units GluR5 to GluR7, and KA-1 and KA-2. Wong and Mayer, Molecular Pharmacology 44: 505-510, 1993. It is not yet known how these subunits are combined in the natural state. However, the structures of certain human variants of each sub-unit have been elucidated, and cell lines expressing individual sub-unit variants have been cloned and incorporated into test systems designed to identify compounds which bind to or interact with them, and hence which may modulate their function. Thus, European patent application, publication number EP-A2-0574257 discloses the human sub-unit variants GluR1 B, GluR2B, GluR3A and GluR3B. European patent application, publication number EP-A1-0583917 discloses the human sub-unit variant GluR4B.

One distinctive property of AMPA and kainic acid receptors is their rapid deactivation and desensitization to glutamate. Yamada and Tang, The Journal of Neuroscience, September 1993, 13(9): 3904-3915 and Kathryn M. Partin, J. Neuroscience, Nov. 1, 1996, 16(21): 6634-6647.

It is known that the rapid desensitization and deactivation of AMPA and/or kainic acid receptors to glutamate may be inhibited using certain compounds. This action of these compounds is often referred to in the alternative as “potentiation” of the receptors. One such compound, which selectively potentiates AMPA receptor function, is cyclothiazide. Partin et al., Neuron. Vol. 11, 1069-1082, 1993.

International Patent Application Publication WO 98/33496 published Aug. 6, 1998 discloses certain sulfonamide derivatives which are useful, for example, for treating psychiatric and neurological disorders, for example cognitive disorders; neuro-degenerative disorders such as Alzheimer's disease; age-related dementias; age-induced memory impairment; movement disorders such as tardive dyskinesia, Huntington's chorea, myoclonus, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, and drug-induced psychosis.

### SUMMARY OF THE INVENTION

The present invention provides compounds of formula I:

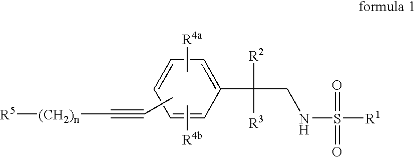

wherein

R¹ represents (1-6C)alkyl, (2-6C)alkenyl, or NR⁶R⁷;

R² and R³ each independently represent hydrogen, (1-4C)alkyl, F, or —OR⁸;

R^{4a} and R^{4b} each independently represent hydrogen, (1-4C) alkyl, (1-4C)alkoxy, I, Br, Cl, or F;

R⁵ represents hydrogen, (1-4C)alkyl, CF₃, OH, OCOCF₃, NH₂, NHCONR¹⁰R¹¹, NHCOR⁹, or NHSO₂R⁹;

n is an integer 1, 2, 3, 4, 5; or 6;

R⁶ and R⁷ each independently represent hydrogen or (1-4C)alkyl;

R⁸ represents hydrogen or (1-4C)alkyl;

R⁹ represents (1-6C)alkyl or NR⁶R⁷; and

R¹⁰ and R¹¹ each independently represent hydrogen or (1-4C)alkyl;

or a pharmaceutically acceptable salt thereof.

The present invention further provides a method of potentiating glutamate receptor function in a patient, which comprises administering to said patient an effective amount of a compound of formula I.

The present invention provides a method of treating cognitive disorders in a patient, which comprises administering to said patient an effective amount of a compound of formula I.

In addition, the present invention further provides a method of treating cognitive deficits associated with psychosis in a patient, which comprises administering to said patient an effective amount of a compound of formula I.

According to another aspect, the present invention provides the use of a compound of formula I, or a pharmaceutically acceptable salt thereof for the manufacture of a medicament for potentiating glutamate receptor function.

In addition, the present invention provides the use of a compound of formula I or a pharmaceutically acceptable salt thereof for potentiating glutamate receptor function.

The invention further provides pharmaceutical compositions comprising, a compound of formula I and a pharmaceutically acceptable diluent or carrier.

This invention also encompasses novel intermediates, and processes for the synthesis of the compounds of formula I.

### DETAILED DESCRIPTION OF THE INVENTION

In this specification, the term “potentiating glutamate receptor function” refers to any increased responsiveness of glutamate receptors, for example AMPA receptors, to glutamate or an agonist, and includes but is not limited to inhibition of rapid desensitization or deactivation of AMPA receptors to glutamate.

A wide variety of conditions may be treated or prevented by compounds of formula I and their pharmaceutically acceptable salts through their action as potentiators of glutamate receptor function. Such conditions include those associated with glutamate hypofunction, such as psychiatric and neurological disorders, for example cognitive disorders and neuro-degenerative disorders such as Alzheimer's disease; age-related dementias; age-induced memory impairment; cognitive deficits due to autism, Down's syndrome and other central nervous system disorders with childhood onset, cognitive deficits post electroconvulsive therapy, movement disorders such as tardive dyskinesia, Huntington's chorea, myoclonus, dystonia, spasticity, and Parkinson's disease; reversal of drug-induced states (such as cocaine, amphetamines, alcohol-induced states); depression; attention deficit disorder; attention deficit hyperactivity disorder; psychosis; cognitive deficits associated with psychosis, drug-induced psychosis, stroke, and sexual dysfunction. Compounds of formula I may also be useful for improving memory (both short term and long term) and learning ability. The present invention provides the use of compounds of formula I for the treatment of each of these conditions.

The present invention includes the pharmaceutically acceptable salts of the compounds defined by formula I. A compound of this invention can possess a sufficiently acidic group, a sufficiently basic group, or both functional groups, and accordingly react with any of a number of organic and inorganic bases, and inorganic and organic acids, to form a pharmaceutically acceptable salt. The term “pharmaceutically acceptable salt” as used herein, refers to salts of the compounds of the above formula which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts. Such salts include the pharmaceutically acceptable salts listed in Journal of Pharmaceutical Science, 66, 2-19 (1977) which are known to the skilled artisan.

Acids commonly employed to form acid addition salts are inorganic acids is such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, hydrobromide, iodide, acetate, propionate, decanoate, caprate, caprylate, acrylate, ascorbate, formate, hydrochloride, dihydrochloride, isobutyrate, caproate, heptanoate, propiolate, propionate, phenylpropionate, salicylate, oxalate, malonate, succinate, suberate, sebacate, fumarate, malate, maleate, hydroxymaleate, mandelate, nicotinate, isonicotinate, cinnamate, hippurate, nitrate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydroxybenzoate, methoxybenzoate, dinitrobenzoate, oacetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, benzenesulfonate, methanesulfonate, ethanesulfonate, propanesulfonate, hydroxyethanesulfonate, 1-naphthalenesulfonate, 2-napththalenesulfonate, 1,5-naphthalenedisulfonate, mandelate, tartarate, and the like. Preferred pharmaceutically acceptable acid addition salts are those formed with mineral acids such as hydrochloric acid and hydrobromic acid, and those formed with organic acids such as maleic acid, oxalic acid and methanesulfonic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that the above salts may form hydrates or exist in a substantially anhydrous form.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ee = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 70:30 is achieved, the ee with respect to the first enantiomer is 40%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the is appropriate chiral column, eluent and conditions necessary to effect separation of the enantiomeric pair is well within the knowledge of one of ordinary skill in the art. In addition, the specific stereoisomers and enantiomers of compounds of formula I can be prepared by one of ordinary skill in the art utilizing well known techniques and processes, such as those disclosed by J. Jacques, et al., “Enantiomers, Racemates, and Resolutions”, John Wiley and Sons, Inc., 1981, and E. L. Eliel and S. H. Wilen, “Stereochemistry of Organic Compounds”, (Wiley-Interscience 1994), and European Patent Application No. EP-A-838448, published Apr. 29, 1998. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

As used herein, the term “aromatic group” means the same as aryl, and includes phenyl and a polycyclic aromatic carbocyclic ring such as 1- or 2-naphthyl, 1,2-dihydronaphthyl, 1,2,3,4-tetrahydronaphthyl, and the like.

The term “heteroaromatic group” includes an aromatic 5-6 membered ring containing from one to four heteroatoms selected from oxygen, sulfur and nitrogen, and a bicyclic group consisting of a 5-6 membered ring containing from one to four heteroatoms selected from oxygen, sulfur and nitrogen fused with a benzene ring or another 5-6 membered ring containing one to four atoms selected from oxygen, sulfur and nitrogen. Examples of heteroaromatic groups are thienyl, furyl, oxazolyl, isoxazolyl, oxadiazoyl, pyrazolyl, thiazolyl, thiadiazolyl, isothiazolyl, imidazolyl, triazolyl, tetrazolyl, pyridyl, pyridazinyl, pyrimidyl, benzofuryl, benzothienyl, benzimidazolyl, benzoxazolyl, benzothiazolyl, indolyl, and quinolyl.

The term “substituted” as used in the term “substituted aromatic or heteroaromatic group” herein signifies that one or more (for example one or two) substituents may be present, said substituents being selected from atoms and groups which, when present in the compound of formula I, do not prevent the compound of formula I from functioning as a potentiator of glutamate receptor function.

Examples of substituents which may be present in a substituted aromatic or heteroaromatic group include I, Br, Cl, F, NH₂, NO₂, cyano; (1-6C) alkyl, (1-6C)alkoxy, (2-6C)alkenyl; (2-6C)alkynyl; (3-8C)cycloalkyl; halo(1-6C)alkyl, and the like.

The term (1-6C)alkyl refers to a straight or branched alkyl chain having from one to six carbon atoms and includes (1-4C)alkyl. Particular values are methyl, ethyl, propyl, isopropyl, butyl, isobutyl, t-butyl, pentyl, and hexyl.

The terms “halogen”, “Hal” or “halide” include fluorine, chlorine, bromine and iodine unless otherwise specified.

The term (1-6C)alkoxy, refers to a straight or branched alkyl chain having from one to six carbon atoms attached to an oxygen atom and includes (1-4C)alkoxy, such as methoxy, ethoxy, propoxy, isopropoxy, isobutoxy, tert-butoxy, pentoxy, and the like.

The compounds of formula I can be prepared by one of ordinary skill in the art, for example, following the procedures set forth below in the Schemes below. The reagents and starting materials are readily available to one of ordinary skill in the art. All substituents, unless otherwise specified are as previously defined.

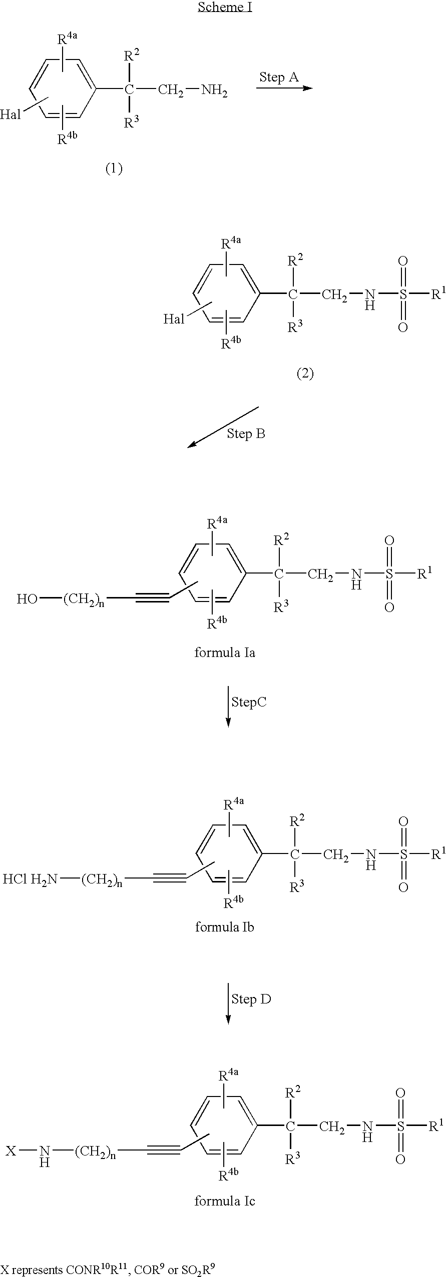

In Scheme 1, step A, the compound of structure (1) is converted to the sulfonamide of structure (2) under conditions well known in the art. More specifically, for example, the compound (1) is dissolved in a suitable organic solvent. Examples of suitable organic solvents include methylene chloride, tetrahydrofuran, and the like. The solution is treated with a slight excess of a suitable base, and then cooled to about −78° C. to about 0° C. Examples of suitable bases include triethylamine, pyridine, 1,8-diazabicyclo[5.4.0]undec-7-ene (DBU), and the like. To the stirring solution is added one equivalent of LgSO₂R¹.

The term “Lg” as used herein refers to a suitable leaving group. Examples of suitable leaving groups include, Cl, Br, and the like. Cl is the preferred leaving group. The reaction mixture is stirred at about 0° C. to about 50° C. for about 0.5 hours to about 16 hours. The compound (2) is then isolated and purified by techniques well known in the art, such as extraction techniques and chromatography. For example, the mixture is washed with 10% sodium bisulfate, the layers separated and the aqueous extracted with several times with a suitable organic solvent, such as methylene chloride. The organic extracts are combined, dried over anhydrous sodium sulfate, filtered and concentrated under vacuum. The residue is then purified by flash chromatography on silica gel with a suitable eluent such as ethyl acetate/hexane to provide the compound (2).

In Scheme I, step B, compound (2) is coupled with an acetylenic alcohol of formula HO(CH₂)ₙCCH under conditions well known in the art such as those described by Sonogashira, Todha and Hagihara, Tetrahedron Lett., 4467-4470, (1975) and improved by Thorand and Krause, J. Org. Chem., 63, 8551-8553 (1998) to provide the compound of formula Ia. More specifically, for example, compound (2) is combined with about 1.1 equivalents of acetylenic alcohol HO(CH₂)ₙCCH with a catalytic amount of a suitable catalyst, such as Pd(PPh₃)₂ in a suitable organic solvent, such as THF under a nitrogen atmosphere. To this solution is then added about 2 equivalents of triethylamine and a catalytic amount of CuI. The reaction mixture is stirred for about 8 to 16 hours and the compound of formula Ia is isolated and purified by techniques well known in the art. For example, the reaction mixture is diluted with a suitable organic solvent, such as diethyl ether, rinsed with water, brine, the organic phase dried over anhydrous magnesium sulfate, filtered and concentrated under vacuum to provide the crude compound of formula Ia. This crude material can then be purified by chromatography on silica gel with a suitable eluent, such as ethyl acetate/hexane.

In Scheme I, step C, the compound of formula Ia is converted directly to the azide which is then converted to the amine of formula Ib under standard conditions. Alternatively, the compound of formula Ia can be converted to the trifluoroacetate ester and then reacted with NaN₃ to provide the intermediate azide. The intermediate azide can then be converted to the amine under standard conditions. For example, see WO 99/26927, published Jun. 3, 1999.

More specifically, the compound of formula Ia is combined with about 1.1 equivalents of triphenylphosphine, about 1.1 equivalents of HN₃, and about 1.1 equivalents of diethyl azodicarboxylate in a suitable organic solvent, such as THF. The reaction mixture is allowed to stir for about 1 to 3 hours and the solvent is then removed under vacuum to provide the crude azide intermediate. This crude material can then be purified by chromatography on silica gel with a suitable eluent, such as ethyl acetate/hexane. This azide intermediate is then combined with about 1.2 equivalents of triphenylphosphine in a mixture of THF/water (10:1). The mixture is heated at a gentle reflux for about 24 to 48 hours. After cooling, concentrated HCl is added and the reaction mixture is concentrated under vacuum to provide the crude compound of formula Ib. This crude material can then be purified by trituration with a suitable organic solvent, such as ethanol.

In Scheme I, step D, the compound of formula Ib is converted to the compound of formula Ic under conditions well known in the art. For example, when X represents —COR⁹, —CONR¹⁰R¹¹ or —SO₂R⁹, the compound of formula Ib is dissolved in a suitable organic solvent, such as methylene chloride and treated with about 2.5 equivalents of triethylamine and about 1.5 equivalents of an acid halide of formula Hal-COR⁹, a carbamyl halide of formula Hal-CONR¹⁰R¹¹, or a sulfonyl halide of formula Hal-SO₂R⁹, wherein Hal preferably represents Cl. The reaction mixture is stirred for about 4 to 16 hours and the crude product is isolated and purified by techniques well known in the art. For example, the solvent can be removed under vacuum and the crude residue purified by chromatography on silica gel with a suitable eluent such as ethyl acetate/hexane to provide the purified compound of formula Ic.

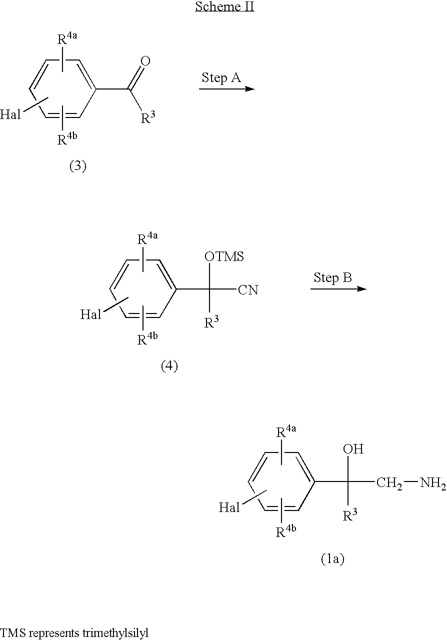

In Scheme II, step A the compound of structure (3) is converted to the compound of structure (4) under standard conditions. For example, see Greenlee and Hangauer, Tetrahedron Lett., 24(42), 4559 (1983). For example, compound (11) is dissolved in a suitable organic solvent, such as dry tetrahydrofuran, containing excess 18-crown-6, and excess potassium cyanide. To this mixture at room temperature is added dropwise about 1.2 equivalents of cyanotrimethylsilane. The reaction mixture is allowed to stir for about 1 to 4 hours to provide compound (4). Compound (4) is then carried on directly to step B without isolation.

Alternatively, in Scheme II, step A, for example, compound (3) is combined with a catalytic amount of zinc iodide followed by slow addition of excess trimethylsilyl cyanide with the generation of heat. The resulting solution is stirred at room temperature under nitrogen for about 8 to 16 hours. The mixture is then diluted with a suitable organic solvent, such as chloroform, washed with saturated sodium bicarbonate, water, brine, dried over anhydrous magnesium sulfate, filtered, and concentrated under vacuum to provide compound (4).

In Scheme II, step B compound (4) is converted to compound of structure (1a). For example, compound (4) prepared above, still in solution, is treated with a solution of about 1.4 equivalents of borane in dimethylsulfide. The reaction mixture is then heated to reflux for about 16 hours and then cooled to room temperature. The reaction mixture is then cautiously treated with anhydrous HCl in methanol and allowed to stir for about one hour. The product (1a) is then isolated and purified using standard techniques and procedures. For example, the solvent is removed under vacuum and the residue triturated with a suitable organic solvent, such at methy t-butyl ether and the solid is collected by filtration.

The solid is then suspended in methylene chloride/tetrahydrofuran mixture (1:2.4) and treated with 1 N NaOH until about pH 12.3 is reached. The phases are separated and the organic phase is rinsed with brine. The organic phase is then concentrated under vacuum and the residue triturated with diethyl ether to provide the purified compound (1a).

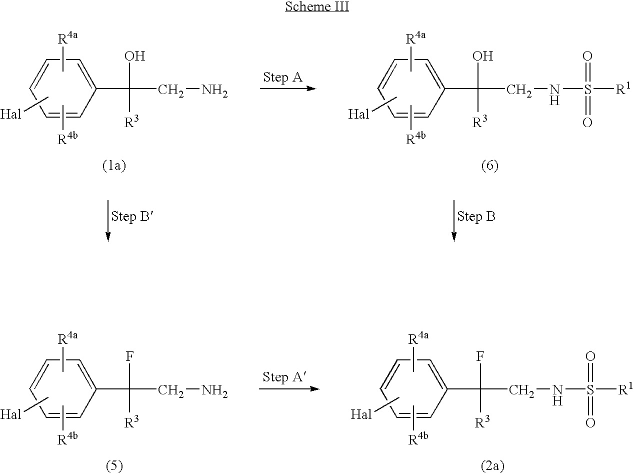

In Scheme III, step A the compound of structure (1a) is combined with a compound of structure LgSO₂R¹ under conditions well known in the art to provide the compound of structure (6). More specifically, for example, the compound (1a) is dissolved in a suitable organic solvent. Examples of suitable organic solvents include methylene chloride, tetrahydrofuran, and the like. The solution is treated with a slight excess of a suitable base, and then cooled to about −78° C. to about 0° C. Examples of suitable bases include triethylamine, pyridine, 1,8-diazabicyclo[5.4.0]undec-7-ene (DBU), and the like. To the stirring solution is added one equivalent of LgSO₂R¹. The term “Lg” as used herein refers to a suitable leaving group. Examples of suitable leaving groups include, Cl, Br, and the like. Cl is the preferred leaving group. The reaction mixture is stirred at about 0° C. to about 50° C. for about 0.5 hours to about 16 hours. The compound (6) is then isolated and purified by techniques well known in the art, such as extraction techniques and chromatography. For example, the mixture is washed with 10% sodium bisulfate, the layers separated and the aqueous extracted with several times with a suitable organic solvent, such as methylene chloride. The organic extracts are combined, dried over anhydrous sodium sulfate, filtered and concentrated under vacuum. The residue is then purified by flash chromatography on silica gel with a suitable eluent such as ethyl acetate/hexane to provide the compound (6).

In Scheme III, step B the compound of structure (6) is fluorinated under conditions well known in the art to provide the compound of structure (2a). For example, compound (6) is dissolved in a suitable organic solvent, such as methylene chloride and the solution is cooled to about −78° C. under an inert atmosphere, such as nitrogen. To this solution is added slowly, about one equivalent of diethylaminosulfur trifluoride (DAST) dissolved in a suitable organic solvent, such as methylene chloride with stirring. The reaction is then allowed to warm to room temperature (about 22° C.) and the compound (2a) is then isolated and purified using techniques and procedures well known in the art, such as extraction techniques and chromatography. For example, the reaction is diluted with water and methylene chloride. The layers are separated and the organic layer is washed with water, dried over anhydrous sodium sulfate, filtered and concentrated under vacuum to provide the crude compound (2a). This crude material can then be purified by standard techniques, such as recrystallization from a suitable eluent, or flash chromatography or radial chromatography (radial chromatography is carried out using a Chromatotron®, Harrison Research Inc., 840 Moana Court, Palo Alto Calif. 94306) on silica gel, with a suitable eluent, such as hexane/ethyl acetate to provide purified compound (2a).

Alternatively, in Scheme III, step B′ the compound (1a) is fluorinated in a manner analogous to the procedure described in step B above with DAST to provide the compound of structure (5). In Scheme III, step A′ compound (5) is converted to compound (2a) in a manner analogous to the procedure described in step A above.

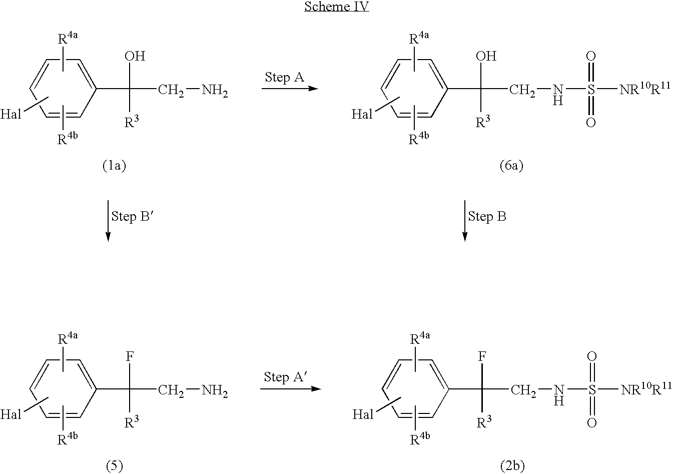

In Scheme IV, step A, the compound of structure (1a) is combined with a compound of formula ClSO₂NR¹⁰R¹¹ under standard conditions to provide the compound of structure (6a). For example, compound (1a) is dissolved in a suitable organic solvent, such as tetrahydrofuran and treated with about one equivalent of a suitable base, such as DBU at about 0° C. The solution is then treated with about one equivalent of a compound of formula ClSO₂NR¹⁰R¹¹. The reaction is then allowed to warm to room temperature and stirred for about 4 to 16 hours. The reaction is then concentrated under vacuum to provide the crude product (6a) which can then be purified by chromatography on silica gel with a suitable eluent, such as ethyl acetate/hexane.

In Scheme IV, step B, compound (6a) is converted to the compound of structure (2b) in a manner analogous to the procedure set forth in Scheme III, step B.

Alternatively, in Scheme IV, step B′ the compound (1a) is fluorinated in a manner analogous to the procedure described in Scheme III, step B with DAST to provide the compound of structure (5). In Scheme IV, step A′ compound (5) is converted to the compound of structure (2b) in a manner analogous to the procedure described above in step A.

The following examples further illustrate the invention and represent typical syntheses of the compounds of formula I as described generally above. The reagents and starting materials are readily available to one of ordinary skill in the art. As used herein the term “Chromatotron®” (Harrison Research Inc., 840 Moana Court, Palo Alto Calif. 94306) is recognized by one of ordinary skill in the art as an instrument which is used to perform centrifugal thin-layer chromatography. As used herein, the following terms have the meanings indicated: “eq” refers to equivalents; “g” refers to grams; “mg” refers to milligrams; “kPa” refers to kilopascals; “L” refers to liters; “mL” refers to milliliters; “μL” refers to microliters; “mol” refers to moles; “mmol” refers to millimoles; “psi” refers to pounds per square inch; “min” refers to minutes; “h” or “hr” refers to hours; “° C.” refers to degrees Celsius; “TLC” refers to thin layer chromatography; “HPLC” refers to high performance liquid chromatography; “Rf” refers to retention factor; “Rₜ” refers to retention time; “δ” refers to part per million down-field from tetramethylsilane; “THF” refers to tetrahydrofuran; “DMF” refers to N,N-dimethylformamide; “DMSO” refers to methyl sulfoxide; “LDA” refers to lithium diisopropylamide; “EtOAc” refers to ethyl acetate; “aq” refers to aqueous; “iPrOAc” refers to isopropyl acetate; “PdCl₂(dppf)” refers to [1,1′bis(diphenylphosphino)ferrocene] dichloropalladium (II); “Ph” refers to phenyl; “PPh₃” refers to triphenylphosphine; “DEAD” refers to diethyl azodicarboxylate; “methyl DAST” refers to dimethylaminosulfur trifluoride, “DAST” refers to diethylaminosulfur trifluoride, “DBU” refers to 1,8-diazabicyclo[5.4.0]undec-7-ene; “TFA” refers to trifluoroacetic acid; “DME” refers to dimethoxyethane; “9-BBN” refers 9-borabicyclo[3.3.1]nonane; and “RT” refers to room temperature.

### Preparation 1

Preparation of [(2R)-2-(4-iodophenyl)propyl][(methylethyl)sulfonyl]amine

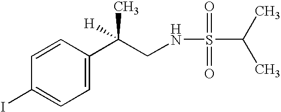

Preparation of 2-Phenyl-1-propylamine HCl.

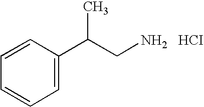

To an autoclave hydrogenation apparatus under nitrogen was charged water-wet 5% palladium on carbon (453 g), ethanol (6.36 L), 2-phenylpropionitrile (636 g, 4.85 moles) and finally concentrated (12M) hydrochloric acid (613 g, 5.6 mole). The mixture was stirred rapidly and pressurized to 75-78 psi with hydrogen. The mixture was then heated to 50-64° C. for 3 hours. ¹H NMR analysis of an aliquot showed less than 5% starting material. The reaction mixture was depressurized and filtered to afford two lots of filtrate that were concentrated under reduced pressure to ˜400 mL each. To each lot was added methyl tert-butyl ether (MTBE) (2.2 L each) and the precipitate solids were allowed to stir overnight. Each lot was filtered and the collected solids were each washed with fresh MTBE (100 mL) and dried overnight. The lots were combined to afford 2-phenyl-1-propylamine HCl (634.4 g, 76.2%) as a white powder.

¹H NMR analysis of the free base: ¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

Preparation of (2R)-2-phenylpropylamine malate.

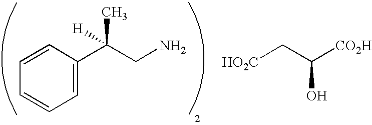

To a dry 3-Liter round bottom flask under nitrogen was charged 2-phenyl-1-propylamine HCl (317.2 g, 1.85 moles), dry ethanol (2.0 L) and NaOH beads (75.4 g, 1.89 moles) that were washed in with additional ethanol (500 mL). The mixture was stirred for 1.6 hours, and the resulting milky white NaCl salts were filtered. An aliquot of the filtrate was analyzed by gas chromatography to provide the amount of free amine, 2-phenyl-1-propylamine, (1.85 moles). A solution of L-malic acid (62.0 g, 0.462 mole, 0.25 equivalents) in ethanol (320 mL) was added dropwise to the yellow filtrate and the solution was heated to 75° C. The solution was stirred at 75° C. for 30 minutes. The heat was removed and the solution was allowed to cool slowly. The resulting thick precipitate was allowed to stir overnight. The precipitate was filtered and dried under vacuum after rinsing with ethanol (325 mL) to afford (2R)-2-phenylpropylamine malate (147.6 g, 39.5%) as a white crystalline solid. Chiral GC analysis of the free base, 2-phenyl-1-propylamine revealed 83.2% e.e. enriched in the R-isomer (configuration was assigned via spectrometric comparison with commercial 2-phenyl-1-propylamine)

¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

A slurry of (2R)-2-phenylpropylamine malate (147.1 g, 83.2% e.e.) in 1325 mL ethanol and 150 mL deionized water was heated to reflux (˜79.2° C.) until the solids went into solution. The homogeneous solution was allowed to slowly cool with stirring overnight. The precipitated white solids were cooled (0-5° C.) and filtered. The collected solids were rinsed with ethanol (150 mL) and dried at 35° C. to afford (2R)-2-phenylpropylamine malate (125.3 g, 85.2% recovery) as a white powder. Chiral GC analysis of the free base, (2R)-2-phenylpropylamine, revealed 96.7% e.e. enriched in the R-isomer.

¹H NMR (CD₃OD, 300 MHz) δ 7.32 (m, 10H), 4.26 (dd, 1H, J=3.6, 9.9), 3.08 (m, 6H), 2.72 (dd, 1H, J=9.3, 15.3), 2.38 (dd, 1H, J=9.3, 15.6), 1.33 (d, 6H, J=6.6).

Preparation of ((2R)-2-phenylpropyl)[(methylethyl)sulfonyl]amine.

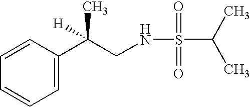

To a stirred slurry of (2R)-2-phenylpropylamine malate (200 g, 0.494 mol) in CH₂Cl₂ (1000 mL) was added 1.0 N NaOH (1050 mL, 1.05 moles). The mixture was stirred at room temperature for 1 hour and the organic phase was separated and gravity filtered into a 3.0 L round-bottom flask with a CH₂Cl₂ rinse (200 mL). The resulting free base, (2R)-2-phenylpropylamine, was dried via azeotropic distillation. Accordingly, the clear filtrate was concentrated to 600 mL at atmospheric pressure via distillation through a simple distillation head. Heptane (1000 mL) was added and the solution was concentrated again at atmospheric pressure to 600 mL using a nitrogen purge to increase the rate of distillation. The final pot temperature was 109° C.

The solution was cooled to room temperature under nitrogen with stirring to give a clear, colorless heptane solution (600 mL) of (2R)-2-phenylpropylamine. To this solution was added 4-dimethylaminopyridine (6.04 g, 0.0494 mol), triethylamine (200 g, 1.98 moles), and CH₂Cl₂ (500 mL). The mixture was stirred at room temperature until a clear solution was obtained. This solution was cooled to 5° C. and a solution of isopropylsulfonyl chloride (148 g, 1.04 moles) in CH₂Cl₂ (250 mL) was added dropwise with stirring over 2 hrs. The mixture was allowed to warm gradually to room temperature over 16 h. GC analysis indicated complete consumption of the (2R)-2-phenylpropylamine starting material.

The stirred mixture was cooled to 8° C. and 2 N HCl (500 mL) was added dropwise. The organic phase was separated and extracted with water (1×500 mL) and saturated NaHCO₃ (1×500 mL). The organic phase was isolated, dried (Na₂SO₄), and gravity filtered. The filtrate was concentrated under reduced pressure to provide ((2R)-2-phenylpropyl)[(methylethyl)sulfonyl]amine (230 g, 96%) as a pale yellow oil. ¹H NMR (CDCl₃, 300 MHz) δ 7.34 (m, 2H), 7.23 (m, 3H), 3.89 (br t, 1H, J=5.4), 3.36 (m, 1H), 3.22 (m, 1H), 3.05 (m, 1H), 2.98 (m, 1H), 1.30 (d, 3H, J=7.2), 1.29 (d, 3H, J=6.9), 1.25 (d, 3H, J=6.9).

Preparation of Final Title Compound.

A stirred room temperature solution of ((2R)-2-phenylpropyl)[(methylethyl)sulfonyl]amine (37.1 g, 0.154 mol) in glacial acetic acid (185 mL) was treated with concentrated H₂SO₄ (16.0 g, 0.163 mol), added dropwise in a slow stream, followed by a H₂O rinse (37 mL). To this solution (˜30° C.) was added H₅IO₆ (8.29 g, 0.0369 mol), followed by iodine (17.9 g, 0.0707 mol). The resulting reaction mixture was heated and allowed to stir for 3 h at 60° C. After HPLC analysis verified the consumption of starting material, the reaction mixture was cooled to 30° C. and a 10% aqueous solution of NaHSO₃ (220 mL) was added dropwise while maintaining the temperature between 25° C. and 30° C. The mixture crystallized to a solid mass upon cooling to 0-5° C.

The solids were suction filtered and rinsed with H₂O to afford 61.7 g of crude solids that were redissolved into warm MTBE (500 mL). This solution was extracted with H₂O (2×200 mL) and saturated NaHCO₃ (1×200 mL) and the organic phase was dried (MgSO₄), filtered, and concentrated under reduced pressure to ˜200 mL. Heptane (100 mL) was added dropwise to the product solution with slow stirring until crystallization commenced. An additional 100 mL of heptane was added and the resulting suspension was allowed to stir slowly overnight at room temperature. The mixture was then cooled (0° C.), filtered, and the collected solids were rinsed with heptane. The solids were then air-dried to afford the final title compound, [(2R{2-(4-iodophenyl)propyl}[(methylethyl)sulfonyl]amine (33.7 g, 59.8%) as a white powder. Chiral Chromatography of this lot indicated 100% e.e.

¹H NMR (CDCl₃, 300 MHz) δ 7.66 (d, 2H, J=8.1), 6.98 (d, 2H, J=8.4), 3.86 (br t, 1H, J=5.1), 3.33 (m, 1H), 3.18 (m, 1H), 3.06 (m, 1H), 2.92 (m, 1H), 1.30 (d, 3H, J=6.6), 1.27 (d, 6H, J=6.6).

### Preparation 2

Preparation of [(2S)-2-(4-iodophenyl)propyl][(methylethyl)sulfonyl]amine.

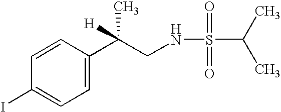

The title compound is prepared from (2S)-2-phenylpropylamine in a manner analogous to the procedure described in preparation 1.

### Preparation 3

Preparation of [2-fluoro-2-(4-iodophenyl)propyl][(methylethyl)sulfonyl]amine.

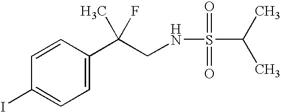

Preparation of 1-amino-2-(4-iodophenyl)Propan-2-ol.

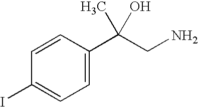

Scheme IV, Step A: The trimethylsilyl-protected cyanohydrin derivative of 4-iodoacetophenone was prepared in situ following generally the method disclosed by Greenlee and Hangauer, Tetrahedron Lett., 24(42), 4559 (1983). Accordingly, cyanotrimethylsilane (21.4 g, 0.216 mol) was added dropwise over 5 minutes to a dry, room temperature solution containing 4-iodoacetophenone (44.3 g, 0.180 mol), 18-crown-6 (1.6 g, 6.1 mmoles) and KCN (1.17g, 0.018 mol) in THF (100 mL). The resulting solution was allowed to stir for 2.5 h. TLC analysis (3:7 EtOAc/Hexanes) showed consumption of starting acetophenone.

Scheme IV, step B: A 10M solution of borane in dimethylsulfide (25 mL, 0.25 mol) was added rapidly to the reaction solution and the resulting mixture was heated at reflux for 16 h. The mixture was cooled to room temperature and anhydrous 10% (by wt) HCl in methanol was added slowly over 1 h (GAS is EVOLUTION). The solution was allowed to stir for an additional hour, and was concentrated under reduced pressure to give the crude title compound as white solid and as the hydrochloride salt. This salt was triturated with methyl t-butyl ether and filtered. The free base was prepared by adding 1N NaOH to a suspension of the HCl salt in CH₂Cl₂ (150 mL) and THF (350 mL) until pH 12.3 was reached. The phases were separated and the organic phase was washed with brine (25 mL). The organic phase containing the free amine was concentrated under reduced pressure and the resulting solids were triturated with diethyl ether (30 mL) to afford the intermediate title compound (35.6 g, 71.3%) as an off-white powder after vacuum drying. ¹H NMR (CD₃OD, 300 MHz): δ 7.68 (d, 2H, J=8.4), 7.24 (d, 2H, J=8.7), 2.78 (m, 2H), 1.46 (s, 3H).

Preparation of [2-hydroxy-2-(4-iodophenyl)propyl][(methylethyl)sulfonyl]amine.

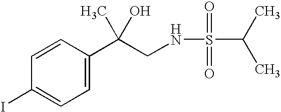

Scheme I, Step A: Into a 250 mL 3 necked flask fitted with a stirrer and thermometer, was added dropwise 2-propanesulfonyl chloride (1.60 g, 0.011 mol) to 1-amino-2-(4-iodophenyl)propan-2-ol (2.77 g, 0.01 mol) in 125 mL CH₂Cl₂ while stirring at 0° C. under nitrogen. The reaction was then allowed to warm to room temperature and stirred overnight at this temperature. In the morning, the mixture was poured into H₂O and the layers were separated. The organic layer was washed once with H₂O, dried over anhydrous Na₂SO₄, filtered, and concentrated under reduced vacuum. The resulting semi-solid was purified via silica gel chromatography employing the Prep. LC-2000 and eluting with a solvent of Hexane/EtOAc 3:1 to provide the intermediate title compound (744 mg, 19%) as a solid material. FDMS 382 (M*).

Analysis for C₁₂H₁₈NO₃ S I:

Theory: C, 37.61 H, 4.73 N, 3.65

Found: C, 38.08 H, 4.26 N, 3.55

Alternative Preparation of Title Compound.

Scheme I, step A: In a 250 mL-3 neck flask fitted with a stirrer and thermometer, 2.10 g. of propanesulfonyl chloride was added dropwise to 2.77 g. of 1-amino-2-(4-iodophenyl)propan-2-ol and 2.30 g. of DBU in CH₂Cl₂ (150 mL) while stirring at 0° C. under a nitrogen atmosphere. The reaction was allowed to warm to room temperature and stirred overnight at this temperature. In the morning, the reaction was diluted with CH₂Cl₂ (100 mL) and the organic layer was washed two times with H₂O, dried over anhydrous Na₂SO₄, filtered, and concentrated under reduced vacuum to yield a viscous oil. This material was purified via silica gel chromatography employing the Chromatotron®, using a 4000 micron rotor and eluting with a solvent of methylene chloride/methanol 19:1 to yield the intermediate title compound (1.0 g, 31%) as a viscous oil. Ion spray M.S. 382 (M*−1).

Preparation of Final Title Compound.

Scheme I, Step B: Into a 10 mL single neck flask, a solution of [2-hydroxy-2-(4-iodophenyl)propyl][(methylethyl)sulfonyl]amine (158 mg, 0.41 mmol) in 1.7 mL CH₂Cl₂ was added syringe wise slowly to a solution of DAST (66 mg, 0.41 mmol) in 0.3 mL CH₂Cl₂ while stirring at −78° C. under nitrogen. The reaction was then allowed to warm to room temperature and the mixture was diluted with H₂O and CH₂Cl₂. The layers were separated and the organic layer was washed twice with H₂O, dried over anhydrous Na₂SO₄, filtered, and concentrated under reduced vacuum to provide the final title compound (113 mg) as a solid. Ion spray M.S. 384 (M*−1).

Additional Preparation of Final Title Compound.

Scheme I, step B: Into a 100 mL 3-neck flask fitted with a stirrer and thermometer, 1.0 g. of [2-hydroxy-2-(4-iodophenyl)propyl][(methylethyl)sulfonyl]amine in CH₂Cl₂ (15 mL) was added dropwise to 0.3 mL DAST in CH₂CL₂ (10 mL) while stirring at −78° C. under a nitrogen atmosphere. Reaction was allowed to warm to room temperature and diluted with CH₂Cl₂ (50 mL). This organic layer was washed with H₂O, dried over anhydrous Na₂SO₄, filtered, and concentrated under reduced vacuum to yield an oil. This material was purified via silica gel chromatography employing the Chromatotron and using a 4000 micron rotor while eluting with a gradient solvent of hexane/ethyl acetate 9:1 to hexane/ethyl acetate 3:1 to yield the final title compound (0.906 g) as a white solid. Ion spray M.S. 384 (M*−1).

Analysis for C₁₂H₁₇NO₂SFI:

Theory: C, 37.42 H, 4.44 N, 3.64

Found: C, 37.27 H, 4.33 N, 3.61

### Preparation 3a

Preparation of (+)-[2-fluoro-2-(4-iodophenyl)propyl][(methylethyl)sulfonyl]amine and (−)-[2-fluoro-2-(4-iodophenyl)propyl][(methylethyl)sulfonyl]amine.

[2-fluoro-2-(4-iodophenyl)propyl][(methylethyl)sulfonyl]amine (2.0 g, prepared in preparation 3) was dissolved into 3A ethanol (30 mL) and was further diluted with heptane (20 mL). [As used herein the term “3A ethanol” refers to ethanol containing 5% methanol.] The mixture was agitated via ultrasound to form a clear, colorless solution. This lot was loaded upon a 8×28 cm preparative Chiralpak AD chromatographic column that was pre-equilibrated with 60% 3A ethanol/40% heptane. Eluent flow was 300 mL/min and detection wavelength was 240 nm. The first eluting substance was (+Y[2-fluoro-2-(4-iodophenyl)propyl][(methylethyl)sulfonyl]amine, [α]_{D}=+18.5 (c=1.08, MeOH), and the subsequent eluting substance was (−)-[2-fluoro-2-(4-iodophenyl)propyl][(methylethyl)sulfonyl]amine, [α]_{D}=−23.5 (c=1.02, MeOH). The above procedure was repeated twice in an analogous manner with [2-fluoro-2-(4-iodophenyl)propyl][(methylethyl)sulfonyl]amine (second run, 3.0 g dissolved in 50 mL 3A ethanol/heptane, 3:2 and a third run, 2.0 g dissolved in 0.8 g dissolved in 40 mL 3A ethanol/heptane, 3:2). Thus, in three runs, a total of 5.8 g of [2-fluoro-2-(4-iodophenyl)propyl][(methylethyl)sulfonyl]amine was resolved into its component enantiomers in the following yields after concentration (in vacuo) of fractions: (+)-[2-fluoro-2-(4-iodophenyl)propyl][(methylethyl)sulfonyl]amine (2.38 g, 41.0%); (−)-[2-fluoro-2-(4-iodophenyl)propyl][(methylethyl)sulfonyl]amine (1.2 g, 20.7%).

Analysis conditions: 0.46×35 cm Chiralpak AD 60% ethanol (5% methanol)/40% Heptane; Flow: 1.0 mL/min, detection wavelength: 240 nm.

For (+Y[2-fluoro-2-(4-iodophenyl)propyl][(methylethyl)sulfonyl]amine: Rₜ=5.4 min, MS (ES+) 384 (M−1).

¹H NMR (CDCl₃, 300 MHz): δ 7.73 (d, 2H, J=8.1), 7.09 (d, 2H, J=8.4), 4.27 (t, 1H, J=6.2), 3.50 (m, 2H), 3.03 (m, 1H), 1.69 (d, 3H, J=22), 1.30 (d, 3H, J=7), 1.27 (d, 3H, J=7).

Analysis for C₁₂H₁₇FINO₂S:

Theory: C, 37.41; H, 4.45; N, 3.64.

Found: C, 37.54; H, 4.43; N, 3.64.

For (−)-[2-fluoro-2-(4-iodophenyl)propyl][(methylethyl)sulfonyl]amine: Rₜ=10.1 min. MS (ES+) 384 (M−1).

¹H NMR spectrum identical to that of (+)-[2-fluoro-2-(4-iodophenyl)propyl][(methylethyl)sulfonyl]amine.

Analysis for C₁₂H₁₇FINO₂S:

Theory: C, 37.41; H, 4.45; N, 3.64.

Found: C, 37.56; H, 4.43; N, 3.59.

### EXAMPLE 1

Preparation of {2-[4-(3-hydroxyprop-1-ynyl)phenyl]propyl}[(methylethyl)sulfonyl]amine.

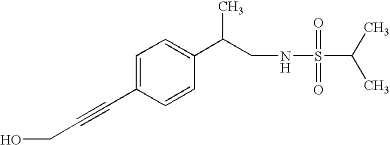

Scheme I, step B: To a stirred solution of [2-(4-iodophenyl)propyl][(methylethyl)sulfonyl]amine (0.50 g, 1.36 mmol), 2-propyn-1-ol (87 μL, 1.50 mmol) and Pd(PPh₃)₂Cl₂ (0.024 g, 0.034 mmol) in dry THF (5.0 mL) was added Et₃N (380 μL, 2.72 mmol) followed by CuI (0.013 g, 0.068 mmol) under nitrogen at ambient temperature. The reaction immediately turned a dark brown color. The brown reaction mixture was stirred overnight at ambient temperature then poured into Et₂O (50 mL) extracted with H₂O (2×50 mL) and washed with brine. The Et₂O layer was separated, dried (MgSO₄), and filtered. Evaporation of the filtrate provided the title compound (0.216 g, 54%) as a brown oil. MS(ES⁺) M+1 296

### EXAMPLE 2

Preparation of {2-[4-(4-hydroxybut-1-ynyl)phenyl]propyl}[(methylethyl)sulfonyl]amine.

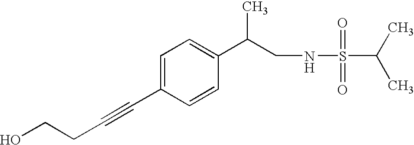

Scheme I, step B: To a stirred solution of [2-(4-iodophenyl)propyl][(methylethyl)sulfonyl]amine (0.50 g, 1.36 mmol), butyn-1-ol (0.114 μL, 1.50 mmol) and Pd(PPh₃)₂Cl₂ (0.024 g, 0.034 mmol) in dry THF (5.0 mL) was added Et₃N (380 μL, 2.72 mmol) followed by CuI (0.013 g, 0.068 mmol) under nitrogen at ambient temperature. The reaction immediately turned a dark brown color. The brown reaction mixture was stirred overnight at ambient temperature then poured into Et₂O (50 mL) extracted with H₂O (2×50 mL) and washed with brine. The Et₂O layer was separated, dried (MgSO₄), and filtered. Evaporation of the filtrate provided the title compound (0.228 g, 54%) as a brown oil. MS(ES⁺⁾ M+1 310

### EXAMPLE 3

Preparation of {2-[4-(5-hydroxypent-1-ynyl)phenyl]propyl}[(methylethyl)sulfonyl]amine.

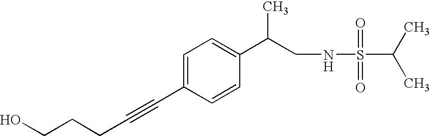

Scheme I, step B: To a stirred solution of [2-(4-iodophenyl)propyl][(methylethyl)sulfonyl]amine (4.00 g, 1.09 mmol), pentyn-1-ol (1.22 mL, 13.1 mmol), and Pd(PPh₃)₂Cl₂(0.200 g, 0.285 mmol) in dry THF (65.0 mL) was added Et₃N (3.36 mL, 24.0 mmol) followed by CuI (0.102 g, 0.285 mmol). The resulting mixture turned a brown color, exothermed slightly and began to deposit a precipitate within 20 minutes. The reaction mixture was stirred overnight under nitrogen at ambient temperature. The resulting suspension was diluted with Et₂O (350 mL) and filtered through Celite®. The yellow filtrate was extracted with H₂O (3×200 mL), washed with brine, separated, and dried (MgSO₄). Filtration, evaporation, and chromatography using a 40S cartridge eluting with EtOAc/hexane (4:6) provided the title compound (2.55 g, 72%) as a colorless oil. MS(ES⁺) M+1 324

### EXAMPLE 4

Preparation of {2-[4-(6-hydroxyhex-1-ynyl)phenyl]propyl}[(methylethyl)sulfonyl]amine.

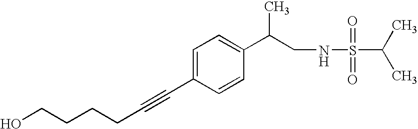

Scheme I, step B: To a stirred solution of [2-(4-iodophenyl)propyl][(methylethyl)sulfonyl]amine (2.00 g, 5.45 mmol), hexyn-1-ol (0.662 mL, 6.00 mmol), and Pd(PPh₃)₂Cl₂ (0.098 g, 0.14 mmol) in dry THF (30 mL) was added Et₃N (1.52 mL, 10.90 mmol) followed by CuI (0.051 g, 0.27 mmol). The resulting mixture turned a brown color. The reaction mixture was stirred overnight under nitrogen at ambient temperature. The resulting suspension was diluted with Et₂O (150 mL) and filtered through Celite®. The yellow filtrate was extracted with H₂O (3×150 mL), washed with brine, separated and dried (MgSO₄). Filtration, evaporation, and chromatography on the Chromatotron® eluting with EtOAc/hexane (1:1) provided the title compound (1.1 g, 59%) as a colorless oil. MS(ES⁺) M+1 338

### EXAMPLE 5

Preparation of {2-[4-(5-aminopent-1-ynyl)phenyl]propyl}[(methylethyl)sulfonyl]amine Hydrochloride.

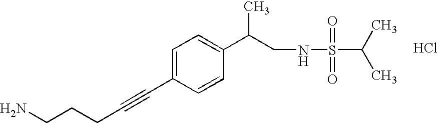

Preparation of {2-[4-(5-azidoinent-1-ynyl)phenyl]propyl}[(methylethyl)sulfonyl]amine.

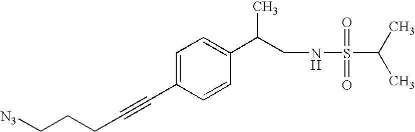

Scheme I, step C: To a stirred mixture of {2-[4-(5-hydroxypent-1-ynyl)phenyl]propyl}[(methylethyl)sulfonyl]amine (0.96 g, 2.96 mmol, prepared in example 3) and PPh₃ (0.855 g, 3.26 mmol) in dry THF (20 mL) was added 0.6M HN₃ (5.4 mL, 3.26 mmol) and DEAD (513 μL, 3.26 mmol). After stirring for 1 hour, the solvent was evaporated in vacuo and the resulting residue was chromatographed on the Chromatotron® with a 2 mm plate (silica gel) eluting with EtOAc/hexane (3:7) to provide the intermediate title compound, {2-[4-(5-azidopent-1-ynyl)phenyl]propyl}[(methylethyl)sulfonyl]amine, as a yellow oil. MS(E⁺): M+18 366

Preparation of Final Title Compound.

Scheme I, step C: {2-[4-(5-Azidopent-1-ynyl)phenyl]propyl}[(methylethyl)sulfonyl]amine (0.96 g, 2.76 mmol) and PPh₃ (0.868 g, 3.31 mmol) were heated and stirred under gentle reflux in a mixture of THF (20 mL) and H₂O (2.0 mL) for 48 hours. Two drops of concentrated HCl were added and the mixture was concentrated to dryness in vacuo. Trituration with hot EtOH and decanting followed by concentration gave a white foam. The foam was dissolved in CH₂Cl₂ and the CH₂Cl₂ was poured into Et₂O precipitating a white solid. The solid was collected by filtration to provide the final title compound, {2-[4-(5-aminopent-1-ynyl)phenyl]propyl}[(methylethyl)sulfonyl]amine hydrochloride. MS(ES) M−1 357; M+1 (FB) 324

### EXAMPLE 6

Preparation of {2-[4-(6-aminohex-1-ynyl)phenyl]propyl}[(methylethyl)sulfonyl]amine Hydrochloride.

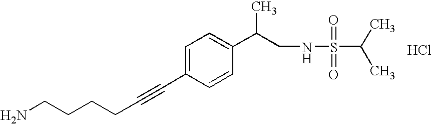

Preparation of {2-[4-(6-azidohex-1-ynyl)phenyl]propyl}[(methylethyl)sulfonyl]amine.

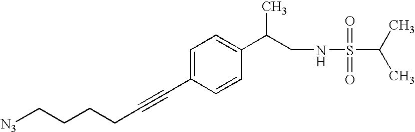

Scheme I, step C: To a stirred mixture of {2-[4-(6-hydroxyhex-1-ynyl)phenyl]propyl}[(methylethyl)sulfonyl]amine (1.0 g, 2.96 mmol, prepared in example 4) and PPh₃ (0.855 g, 3.26 mmol) in dry THF (20 mL) was added 0.6M HN₃ (5.4 mL, 3.26 mmol) and DEAD (513 μL, 3.26 mmol). After stirring for 1 h the solvent was evaporated in vacuo and the resulting residue was chromatographed on a 2 mm plate (silica gel) with the Chromatotron® eluting with EtOAc-hexane 3:7 to provide the title compound (1.0 g, 93%) as a yellow oil. MS(ES⁺):M+18, 380.

Preparation of Final Title Compound.

Scheme I, step C: {2-[4-(6-Azidohex-1-ynyl)phenyl]propyl}[(methylethyl)sulfonyl]amine (0.1.0 g, 2.76 mmol) and PPh₃ (0.868 g, 3.31 mmol) were heated and stirred under gentle reflux in a mixture of THF (20 mL) and H₂O (2.0 mL) for 48 h. Two drops of concentrated HCl were added and the mixture was concentrated to dryness in vacuo. Trituration with hot EtOH and decanting followed by concentration gave a white foam. The foam was dissolved in CH₂Cl₂ and the CH₂Cl₂ was poured into Et₂O precipitating a white solid. The solid was collected by filtration to provide the final title compound (0.510 g, 50%)

Analysis for C₁₀H₂₈N₂SO₂.HCl

Theory: C, 57.97; H, 7.84; N, 7.51

Found; C, 57.73; H, 7.77; N, 7.35

### EXAMPLE 7

Preparation of N-{6-[4-(1-methyl-2-{[(methylethyl)sulfonyl]amino}ethyl)phenyl]hex-5-ynyl}acetamide.

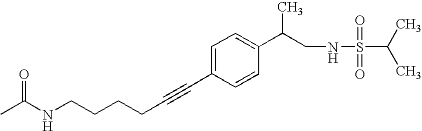

Scheme I, step D: To a stirred solution of {2-[4-(6-aminohex-1-ynyl)phenyl]propyl}[(methylethyl)sulfonyl]amine (0.100 g, 0.27 mmol, prepared in example 6) in CH₂Cl₂ (1.4 mL) was added Et₃N (95 μL, 0.68 mmol) followed by acetyl chloride (29 μL, 0.41 mmol), and the resulting mixture was stirred overnight at ambient temperature. The reaction was chromatographed on a 2 mm plate (silica gel) on the Chromatotron® eluting with EtOAc/hexane 3:7 to provide the title compound as a white powder. MS(ES⁺): M+1 415

### EXAMPLE 8

Preparation of N-{6-[4-(1-methyl-2-{[(methylethyl)sulfonyl]amino}ethyl)phenyl}hex-5-ynyl]methylsulfonamide.

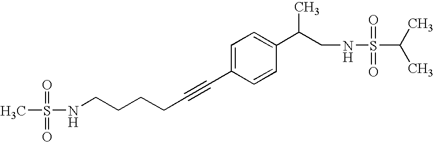

Scheme I, step D: To a stirred solution of {2-[4-(6-aminohex-1-ynyl)phenyl]propyl}[(methylethyl)sulfonyl]amine (0.100 g, 0.27 mmol, prepared in example 6) in CH₂Cl₂ (1.4 mL) was added Et₃N (95 μL, 0.68 mmol) followed by methanesulfonyl chloride (32 μL, 0.41 mmol) and the resulting mixture was stirred overnight at ambient temperature. The reaction was chromatographed on a 2 mm plate (silica gel) on the Chromatotron® eluting with EtOAc/hexane 3:7 to provide the title compound as a white powder.

Analysis for C₁₉H₃₀N₂S₂O₄:

Theory: C, 55.05; H, 7.29; N, 6.76

Found: C, 54.66; H, 7.07; N, 6.62

### EXAMPLE 9

Preparation of (dimethylamino)-N-{6-[4-(1-methyl-2-{[(methylethyl)sulfonyl]amino}ethyl)phenyl]hex-5-ynyl}carboxamide.

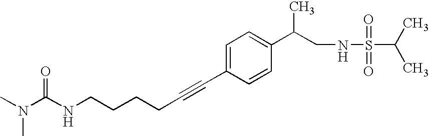

Scheme I, step D: To a stirred solution of {2-[4-(6-aminohex-1-ynyl)phenyl]propyl}[(methylethyl)sulfonyl]amine (0.100 g, 0.27 mmol, prepared in example 6) in CH₂Cl₂ (1.4 mL) was added Et₃N (95 μL, 0.68 mmol) followed by dimethyl carbamyl chloride (36 μL, 0.41 mmol) and the resulting mixture was stirred overnight at ambient temperature. The reaction was chromatographed on a 2 mm plate (silica gel) on the Chromatotron® eluting with EtOAc/hexane 3:7 to provide the title compound as a white powder.

Analysis for C₂₁H₃₃N₃S₂O₃:

Theory: C, 61.89; H, 8.16; N, 10.31

Found: C, 61.82; H, 8.08; N, 10.17

### EXAMPLE 10

Preparation of 2-methyl-N-{6-[4-(1-methyl-2-{[(methylethyl)sulfonyl]amino}ethyl)phenyl]hex-5-ynyl}propanamide.

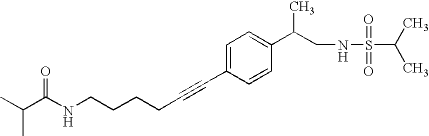

Scheme I, step D: To a stirred solution of {2-[4-(6-aminohex-1-ynyl)phenyl]propyl}[(methylethyl)sulfonyl]amine (0.100 g, 0.27 mmol, prepared in example 6) in CH₂Cl₂ (1.4 mL) was added Et₃N (95 μL, 0.68 mmol) followed by isobutyryl chloride (43 μL, 0.41 mmol) and the resulting mixture was stirred overnight at ambient temperature. The reaction was chromatographed on a 2 mm plate (silica gel) on the Chromatotron® eluting with EtOAc/hexane 1:1 to provide the title compound as a white powder.

Analysis for C₂₂H₃₄N₂S₂O₃:

Theory: C, 64.99; H, 8.43; N, 6.89

Found: C, 65.11; H, 8.40; N, 6.87

### EXAMPLE 11

Preparation of {2-fluoro-2-[4-(6-hydroxyhex-1-ynyl)phenyl]propyl}[(methylethyl)sulfonyl]amine.

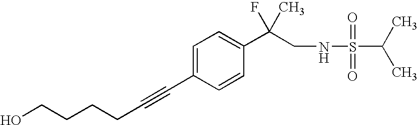

Scheme I, step B: Cuprous iodide (0.019 g, 0.10 mmol) was added to a stirred mixture of [2-fluoro-2-(4-iodophenyl)propyl][(methylethyl)sulfonyl]amine (0.779 g, 2.02 mmol), heyn-1-ol (0.162 g, 2.60 mmol), Et₃N (0.63 mL, 4.5 mmol) and Pd(PPh₃)₂Cl₂ (0.042 g, 0.060 mmol) respectively in THF (15.0 mL) under N₂. The resulting dark brown reaction mixture was stirred at ambient temperature overnight. The reaction mixture was diluted with Et₂O (100 mL) and filtered. The filtrate was extracted with H₂O (2×100 mL) washed with brine and dried (MgSO₄). Filtration and evaporation in vacuo gave a yellow oil which was chromatographed on a 4 mm plate (silica gel) on the Chromatotron® eluting with EtOAc/hexane 4:6 to provide the title compound (0.272 g, 38%) as a colorless oil which solidified on standing overnight, mp 92-93° C.

Analysis for C₁₈H₂₆FNO₃S:

Theory: C, 60.82; H, 7.37; N, 3.91

Found: C, 60.95; H, 7.41; N, 3.98

### EXAMPLE 12

Preparation of 2-[4-(6-amino-hex-1-ynyl)-phenyl]-2-fluoro-propyl]-2-propanesulfonamide Hydrochloride.

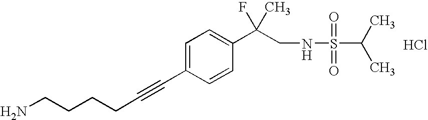

Preparation of 6-{4-[1-fluoro-1-ethyl-2-(propane-2-sulfonylamino)ethyl]-phenyl}-hex-5-ynyl Methane Sulfonic Acid Ester.

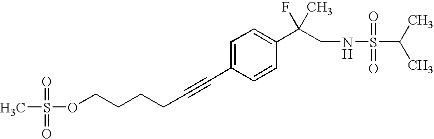

Scheme I, step D: Triethylamine (0.26 mL, 1.85 mmol) was added to a stirred solution of the alcohol from example 11 (0.219 g, 0.62 mmol) and methanesulfonyl chloride (81 μL, 1.23 mmol) in CH₂Cl₂ (18.0 mL) at 0° C. The resulting mixture was stirred at 0 TO 10. for 2 h then overnight at ambient temperature. The mixture was diluted tom 50 mL with CH₂Cl₂ then extracted with H₂O (3×200 mL), washed with brine, separated, dried (MgSO₄) filtered, and the filtrate evaporated to give the desired intermediate title compound (0.218 g, 82%).

Preparation of {2-[4-(6-azido-hex-1-ynyl)-phenyl]-2-fluoro-propyl}-2-propanesulfonamide.

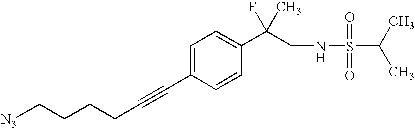

Lithium azide (0.192 g, 3.97 mmol) was added to a stirred solution of 6-{4-[1-fluoro-1-ethyl-2-(propane-2-sulfonylamino)-ethyl]-phenyl}-hex-5-ynyl methane sulfonic acid ester (0.213 g, 0.492 mmol) in dry DMF (20 mL) at ambient temperature under N₂. The resulting mixture was heated and stirred at 80° C. overnight. The DMF mixture was poured into H₂O (200 mL) and extracted with Et₂O (100 mL). The organic layer was separated and extracted with H₂O (3×200 mL). The Et₂O layer was dried, filtered and evaporated to give the intermediate title compound (0.096 g).

Preparation of Final Title Compound.

Triphenyl phosphine (0.071 g, 0.254 mmol) was added to a stirred solution of {2-[4-(6-azido-hex-1-ynyl)phenyl]-2-fluoro-propyl}2-propanesulfonamide (0.096 g, 0.253 mmol) in anhydrous THF (5.0 mL) under N₂ at ambient temperature followed by H₂O (0.5 mL). The reaction mixture was stirred and heated at gentle reflux overnight. Approximately 3 drops of concentrated HCl were added and the reaction mixture was evaporated to dryness. Trituration of the resulting foam with EtOAc gave the final title compound (0.071 g, 72%) as a white solid.

MS(ES) M+1 (FB) 355.2

### EXAMPLE 13

Preparation of 2-fluoro-2-[4-(6-methanesulfonylamino-hex-1-ynyl)-phenyl]propyl-2-propanesulfonamide.

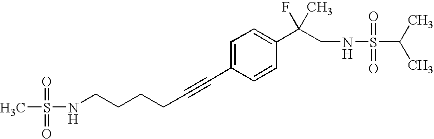

Scheme I, step D: Methanesulfonyl chloride (0.015 mL, 0.200 mmol) was added to a stirred solution of 2-[4-(6-amino-hex-1-ynyl)-phenyl]-2-fluoro-propyl]-2-propanesulfonamide hydrochloride (0.065 g, 0.166 mmol, example 12) and TEA (70 μL, 0.500 mmol) in CH₂Cl₂ (2.0 mL) under N₂ at ambient temperature. The reaction mixture was stirred for 4 h at ambient temperature and additional CH₃SO₂Cl (15 μL) and TEA (70 μL) was added to the cloudy mixture. Everything went into solution and the reaction mixture was stirred overnight at ambient temperature. The reaction mixture was poured into H₂O (150 mL), separated, extracted several times with H₂O, washed with brine, dried, filtered, and evaporated to give a viscous oil. Chromatography on a 1 mm plate on the Chromatotron® eluting with EtOAc/hexane (1:1) gave the title compound (0.037 g) as a colorless oil. MS(ES) M−1 431.2

### EXAMPLE 14

Preparation of (+)-{2-[4-(6-hydroxyhex-1-ynyl)phenyl]propyl}[(methylethyl)sulfonyl]amine.

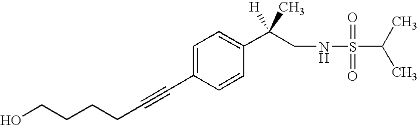

Scheme I, step B: To a stirred solution of (+) [2-(4-iodophenyl)propyl][(methylethyl)sulfonyl]amine (3.81 g, 10.4 mmol), hexyn-1-ol (1.32 g, 13.4 mmol), and Pd(PPh₃)₂Cl₂ (0.22 g, 0.33 mmol) in dry THF (80 mL) was added Et₃N (3.2 mL, 23.0 mmol) followed by CuI (0.10 g, 0.53 mmol). The resulting mixture turned a brown color. The reaction mixture was stirred for 65 h under nitrogen at ambient temperature. The resulting suspension was concentrated on the rotary evaporator and taken up in a mixture o Et₂O (40 mL) and EtOAc (40 mL) and extracted with H₂O (2×40 mL). The organic phase was separated, filtered, and evaporated to give a dark brown oil. Chromatography eluting with EtOAc/hexane (1:1) provided the title compound (1.52 g, 59%) as a yellow oil. MS(ES⁺) M+1 338.15

### EXAMPLE 15

Preparation of (+)-N-{6-[4-(1-methyl-2-{[(methylethyl)sulfonyl]amino}ethyl)phenyl]hex-5-ynyl}trifluoroacetate.

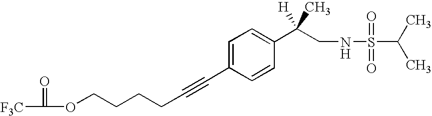

Trifluoroacetic anhydride (2.8 mL, 19.8 mmol) was added to a stirred solution of (+){2-[4-(6-hydroxyhex-1-ynyl)phenyl]propyl}[(methylethyl)sulfonyl]amine (1.35 g, 4.00 mmol, example 14) in CH₂Cl₂ (2.0 mL) at ambient temperature. After 5 min., the reaction was concentrated on the rotary evaporator to provide the title compound (1.66 g, 96%) as a yellow oil. MS(ES) (M−1) 432.91

### EXAMPLE 16

Preparation of {(2R)-2-[4-(6-aminohex-1-ynyl)phenyl]propyl}[(methylethyl)sulfonyl]amine.

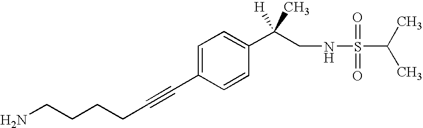

Preparation of (+)-{2-(6-azidohex-1-ynyl)phenyl]propyl}[(methylethyl)sulfonylamine.

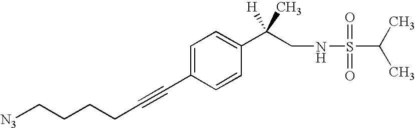

Scheme I, step C: A mixture of (+)N{6-[4-(1-methyl-2-{[(methylethyl)sulfonyl]amino}ethyl)phenyl]hex-5-ynyl}trifluoroacetate (1.60 g, 3.69 mmol, example 15) and NaN₃ (1.20 g, 18.5 mmol) was heated in anhydrous DMF (30 mL) under N₂ at 80° C. for 3 h. TLC showed little product. Additional NaN₃ (1.20 g) was added and the reaction mixture was heated at 100° C. for 20 h. The reaction mixture was allowed to cool to ambient temperature and trifluoroacetic anhydride (0.20 mL, 1.4 mmol) was added and the reaction mixture was heated at 100° C. for 3 h. Additional TFM (0.20 mL) was added at 3 h, 4 h, 5 h, and 6 h (0.10 mL). After 24 h the reaction mixture was poured into H₂O (150 mL) and extracted with Et₂O (50 mL) and then EtOAc (3×50 mL). The organics were combined, washed with H₂O (2×50 mL) and evaporated to give the title compound (0.677 g, 51%) as a cream colored solid.

Analysis for C₁₈H₂₆N₄O₂S

Theory: C, 59.64; H, 7.23; N, 15.46

Found: C, 59.26; H, 7.08; N, 15.09

Preparation of Final Title Compound.

Scheme I, step C: The final title compound can be prepared in a manner analogous to the procedure set forth in example 12 from (+)-{2-(6-azidohex-1-ynyl)phenyl]propyl}[(methylethyl)sulfonylamine.

### EXAMPLE 17

Preparation of N-{6-[4-(1-methyl-2-{[methylethyl)sulfonyl]amino}ethyl)phenyl]pent-4-ynyl}acetamide.

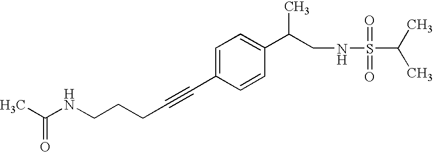

Scheme I, step D: To a stirred solution of {2-[4-(5-aminopent-1-ynyl)phenyl]propyl}[(methylethyl)sulfonyl]amine hydrochloride (0.44 g, 1.12 mmol, example 5) in CH₂Cl₂ (5.0 mL) was added Et₃N (0.78 mL, 5.60 mmol) and acetyl chloride (0.142 mL, 2.0 mmol). The reaction mixture was stirred overnight, diluted with EtOAc, washed with brine, dried (MgSO₄), filtered, and evaporated to provide the title compound (0.212 g).

MS(ES) M+1 365

### EXAMPLE 18

Preparation of N-[6-[-(1-methyl-2-{methylethyl)sulfonyl]amino}ethyl)phenyl]pent-4-ynyl}methyl Sulfonamide.

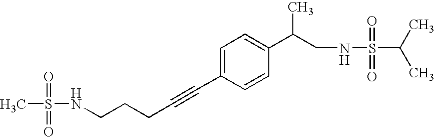

Scheme I, step D: To a stirred solution of {2-[4-(5-aminopent-1-ynyl)phenyl]propyl}[(methylethyl)sulfonyl]amine hydrochloride (0.44 g, 1.12 mmol, example 5) in CH₂Cl₂ (5.0 mL) was added Et₃N (0.78 mL, 5.60 mmol) and methanesulfonyl chloride (0.154 mL, 2.0 mmol). The reaction mixture was stirred overnight, diluted with EtOAc, washed with brine, dried (MgSO₄), filtered, and evaporated to provide the title compound (0.103 g). MS(ES) M+1 401

### EXAMPLE 19

Preparation of N-{6-[4-(1-methyl-2-methylethyl)sulfonyl]amino}ethyl)phenylpent-4-ynyl}tert Butyl Carbamate.

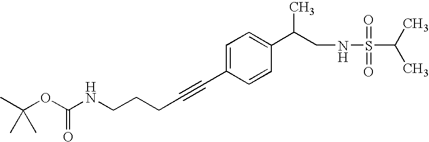

Scheme I, step D: To a stirred solution of {2-[4-(5-aminopent-1-ynyl)phenyl]propyl}[(methylethyl)sulfonyl]amine hydrochloride (0.44 g, 1.12 mmol, example 5) in CH₂Cl₂ (5.0 mL) was added. Et₃N (0.78 mL, 5.60 mmol) and t-butyl dicarbonate (0.459 mL, 2.0 mmol). The reaction mixture was stirred overnight, diluted with EtOAc, washed with brine, dried (MgSO₄), filtered, and evaporated to provide the title compound (0.223 g). MS(ES) M+1 423

### EXAMPLE 20

Preparation of 2-methyl-N-{6-[4-(methyl-2-{[(methylethyl)sulfonyl]amino}ethyl)phenyl]pent-4ynyl}propanamide.

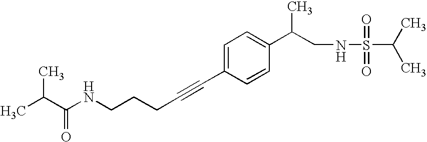

Scheme I, step D: To a stirred solution of {2-[4-(5-aminopent-1-ynyl)phenyl]propyl}[(methylethyl)sulfonyl]amine hydrochloride (0.44 g, 1.12 mmol) in CH₂Cl₂ (5.0 mL) was added Et₃N (0.78 mL, 5.60 mmol) and isobutyl chloroformate (0.209 mL, 2.0 mmol). The reaction mixture was stirred overnight, diluted with EtOAc, washed with brine, dried (MgSO₄), filtered, and evaporated to provide the title compound (0.189 g). MS(ES) M+1 393

### EXAMPLE 21

Preparation of (dimethylamino-N-{6-[4-(1-methyl-2-{[(methylethyl)sulfonyl]amino}ethyl)phenyl]pent-4-ynyl}carboxamide.

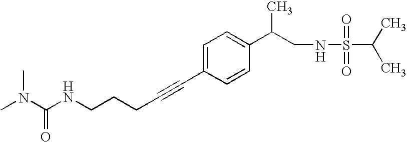

Scheme I, step D: To a stirred solution of {2-[4-(5-aminopent-1-ynyl)phenyl]propyl}[(methylethyl)sulfonyl]amine hydrochloride (0.44 g, 1.12 mmol) in CH₂Cl₂ (5.0 mL) was added Et₃N (0.78 mL, 5.60 mmol) and dimethyl carbamyl chloride (0.183 mL, 2.0 mmol). The reaction mixture was stirred overnight, diluted with EtOAc, washed with brine, dried (MgSO₄), filtered, and evaporated to provide the title compound (0.174 g). MS(ES) M+1 394

### EXAMPLE 22

Preparation of 2-methyl-N-{6-[4-(1-methyl-2-[(methyethyl)sulfonyl]amino}ethyl)phenyl]pent-4-ynyl]ethyl Sulfonamide.

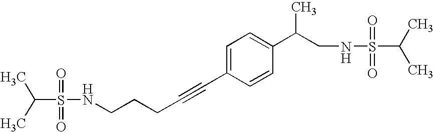

Scheme I, step D: To a stirred solution of {2-[4-(5-aminopent-1-ynyl)phenyl]propyl}[(methylethyl)sulfonyl]amine hydrochloride (0.44 g, 1.12 mmol, example 5) in CH₂Cl₂ (5.0 mL) was added Et₃N (0.78 mL, 5.60 mmol) and isopropyl sulfonyl chloride (0.183 mL, 2.0 mmol). The reaction mixture was stirred overnight, diluted with. EtOAc, washed with brine, dried (MgSO₄), filtered, and evaporated to provide the title compound (0.236 g).

MS(ES) M+1 429

### EXAMPLE 23

Preparation of {2-fluoro-2-[4-(5-hydroxypent-1-ynyl)phenyl]propyl}[(methylethyl)sulfonyl]amine.

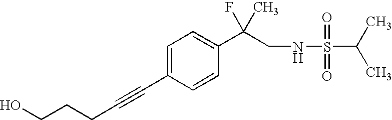

Scheme I, step B: Cuprous iodide (0.0.106 g, 0.56 mmol) was added to a stirred mixture of [2-fluoro-2-(4-iodophenyl)propyl][(methylethyl)sulfonyl]amine (4.0 g, 10.38 mmol), pentyn-1-ol (1.26 mL, 13.54 mmol), Et₃N (3.2 mL, 22.84 mmol) and Pd(PPh₃)₂Cl₂ (0.22 g, 0.030 mmol) respectively in THF (80.0 mL) under N₂. The resulting dark brown reaction mixture was stirred at ambient temperature overnight. The reaction mixture was diluted with Et₂O (100 mL) and filtered. The filtrate was extracted with H₂O (2×100 mL) washed with brine and dried (MgSO₄). Filtration and evaporation in vacuo gave an oil which was chromatographed on a 4 mm plate (silica gel) on the Chromatotron® eluting with EtOAc/2:8 to 7:3 to provide the title compound (2.03 g, 56%) as a yellow oil. MS(ES) M−1 340

### EXAMPLE 24

Preparation of {2-[4-(5-azido-pent-1-ynyl)phenyl]2-fluoro-propyl}-2-propanesulfonamide.

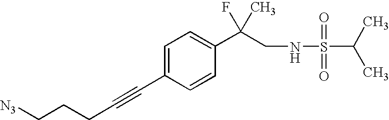

Scheme I, step C: To a stirred mixture of {2-fluoro-2-[4-(5-hydroxypent-1-ynyl)phenyl]propyl}[(methylethyl)sulfonyl]amine (2.0 g, 5.85 mmol, example 23) and PPh₃ (1.61 g, 6.44 mmol) in dry THF (20 mL) was added 0.6 M HN₃ (10.2 mL, 6.44 mmol) and DEAD (0.963 mL, 6.44 mmol). After stirring for 1 hour, the solvent was evaporated in vacuo to provide the title compound as a solid (2.5 g). MS(ES) M−1 365

### EXAMPLE 25

Preparation of {2-[4-(5-aminopent-1-ynyl)phenyl]propyl}[methylethyl)sulfonyl]amine Hydrochloride.

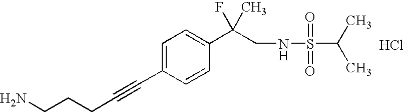

Scheme I, step C: {2-[4-(5-Azido-pent-1-ynyl)phenyl]2-fluoro-propyl}-2-propanesulfonamide (2.5 g, 6.82 mmol, example 24) and PPh₃ (2.14 g, 8.14 mmol) were heated and stirred under gentle reflux in a mixture of THF (20 mL) and H₂O (2.0 mL) for 72 h. Two drops of concentrated HCl were added and the mixture was concentrated to dryness in vacuo. Trituration with hot EtOH and decanting followed by concentration gave a white foam. The foam was dissolved in CH₂Cl₂ and the CH₂Cl₂ was poured into Et₂O precipitating a white solid. The solid was collected by filtration to provide the final title compound (2.0 g, 78%) as a yellow powder. MS(ES) M+1 (FB) 341

The ability of compounds of formula I to potentiate glutamate receptor-mediated response may be determined using fluorescent calcium indicator dyes (Molecular Probes, Eugene, Oreg., Fluo-3) and by measuring glutamate-evoked efflux of calcium into GluR4 transfected HEK293 cells, as described in more detail below.

In one test, 96 well plates containing confluent monolayers of HEK 293 cells stably expressing human GluR4B (obtained as described in European Patent Application Publication Number EP-A1-583917) are prepared. The tissue culture medium in the wells is then discarded, and the wells are each washed once with 200 μl of buffer (glucose, 10 mM, sodium chloride, 138 mM, magnesium chloride, 1 mM, potassium chloride, 5 mM, calcium chloride, 5 mM, N-[2-hydroxyethyl]-piperazine-N-[2-ethanesulfonic acid], 10 mM, to pH 7.1 to 7.3). The plates are then incubated for 60 minutes in the dark with 20 μM Fluo3-AM dye (obtained from Molecular Probes Inc., Eugene, Oreg.) in buffer in each well. After the incubation, each well is washed once with 100 μl buffer, 200 μl of buffer is added and the plates are incubated for 30 minutes.

Solutions for use in the test are also prepared as follows. 30 μM, 10 μM, 3 μM and 1 μM dilutions of test compound are prepared using buffer from a 10 mM solution of test compound in DMSO. 100 μM cyclothiazide solution is prepared by adding 3 μl of 100 mM cyclothiazide to 3 mL of buffer. Control buffer solution is prepared by adding 1.5 μl DMSO to 498.5 μl of buffer.

Each test is then performed as follows. 200 μl of control buffer in each well is discarded and replaced with 45 μl of control buffer solution. A baseline fluorescent measurement is taken using a FLUOROSKAN II fluorimeter (Obtained from Labsystems, Needham Heights, Mass., USA, a Division of Life Sciences International Plc). The buffer is then removed and replaced with 45 μl of buffer and 45 μl of test compound in buffer in appropriate wells. A second fluorescent reading is taken after 5 minutes incubation. 15 μl of 400 μM glutamate solution is then added to each well (final glutamate concentration 100 μM), and a third reading is taken. The activities of test compounds and cyclothiazide solutions are determined by subtracting the second from the third reading (fluorescence due to addition of glutamate in the presence or absence of test compound or cyclothiazide) and are expressed relative to enhance fluorescence produced by 1100 μM cyclothiazide.

In another test, HEK293 cells stably expressing human GluR4 (obtained as described in European Patent Application Publication No. EP-A1-0583917) are used in the electrophysiological characterization of AMPA receptor potentiators. The extracellular recording solution contains (in mM): 140 NaCl, 5 KCl, 10 HEPES, 1 MgCl₂, 2 CaCl₂, 10 glucose, pH=7.4 with NaOH, 295 mOsm kg−1. The intracellular recording solution contains (in mM): 140 CsCl, 1 MgCl₂, 10 HEPES, (N-[2-hydroxyethyl]piperazine-N1-[2-ethanesulfonic acid]) 10 EGTA (ethylene-bis(oxyethylene-nitrilo)tetraacetic acid), pH=7.2 with CsOH, 295 mOsm kg−1. With these solutions, recording pipettes have a resistance of 2-3 MΩ. Using the whole-cell voltage clamp technique (Hamill et al. (1981) Pflügers Arch., 391: 85-100), cells are voltage-clamped at −60 mV and control current responses to 1 mM glutamate are evoked. Responses to 1 mM glutamate are then determined in the presence of test compound. Compounds are deemed active in this test if, at a test concentration of 10 μM or less, they produce a greater than 10% increase in the value of the current evoked by 1 mM glutamate.

In order to determine the potency of test compounds, the concentration of the test compound, both in the bathing solution and co-applied with glutamate, is increased in half log units until the maximum effect was seen. Data collected in this manner are fit to the Hill equation, yielding an EC₅₀ value, indicative of the potency of the test compound. Reversibility of test compound activity is determined by assessing control glutamate 1 mM responses. Once the control responses to the glutamate challenge are re-established, the potentiation of these responses by 100 μM cyclothiazide is determined by its inclusion in both the bathing solution and the glutamate-containing solution. In this manner, the efficacy of the test compound relative to that of cyclothiazide can be determined.

According to another aspect, the present invention provides a pharmaceutical composition, which comprises a compound of formula I or a pharmaceutically acceptable salt thereof and a pharmaceutically acceptable diluent or carrier.

The pharmaceutical compositions are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragcanth, gelatin, calcium silicate, micro-crystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 1 mg to about 500 mg, more preferably about 5 mg to about 300 mg (for example 25 mg) of the active ingredient. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient. The following formulation examples are illustrative only and are not intended to limit the scope of the invention in any way.

### Formulation 1

Hard gelatin capsules are prepared using the following ingredients:

| | Quantify (mg/capsule) |
|---|---|
| Active Ingredient | 250 |
| Starch, dried | 200 |
| Magnesium Stearate | 10 |
| Total | 460 |

The above ingredients are mixed and filled into hard gelatin capsules in 460 mg quantities.

### Formulation 2

Tablets each containing 60 mg of active ingredient are made as follows:

| | Quantity (mg/tablet) |
|---|---|
| Active Ingredient | 60 |
| Starch | 45 |
| Microcrystalline Cellulose | 35 |
| Polyvinylpyrrolidone | 4 |
| Sodium Carboxymethyl Starch | 4.5 |
| Magnesium Stearate | 0.5 |
| Talc | 1 |
| Total | 150 |

As used herein the term “active ingredient” refers to a compound of formula I. The active ingredient, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50° C. and passed through a No. 18 mesh U.S. sieve. The sodium carboxymethyl starch, magnesium stearate, and talc, previously passed through a No. 60 mesh U.S. sieve, are then added to the granules which, after mixing, are compressed on a tablet machine to yield tablets each weighing 150 mg.

As used herein the term “patient” refers to a mammal, such as a mouse, guinea pig, rat, dog or human. It is understood that the preferred patient is a human.

As used herein, the terms “treating” or “to treat” each mean to alleviate symptoms, eliminate the causation either on a temporary or permanent basis, or to prevent or slow the appearance of symptoms of the named disorder. As such, the methods of this invention encompass both therapeutic and prophylactic administration.

As used herein, the term “effective amount” refers to the amount of a compound of formula I which is effective, upon single or multiple dose administration to a patient, in treating the patient suffering from the named disorder.

An effective amount can be readily determined by the attending diagnostician, as one skilled in the art, by the use of known techniques and by observing results obtained under analogous circumstances. In determining the effective amount or dose, a number of factors are considered by the attending diagnostician, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds can be administered by a variety of routes including oral, rectal, transdermal, subcutaneous, intravenous, intramuscular, bucal or intranasal routes. Alternatively, the compound may be administered by continuous infusion. A typical daily dose will contain from about 0.01 mg/kg to about 100 mg/kg of the active compound of this invention. Preferably, daily doses will be about 0.05 mg/kg to about 50 mg/kg, more preferably from about 0.1 mg/kg to about 25 mg/kg.

The compounds of formula I as a class are particularly useful in the treatment methods of the present invention, but certain groups, substituents, and configurations are preferred for compounds of formula I. The following paragraphs describe such preferred groups, substituents, and configurations. It will be understood that these preferences are applicable both to the treatment methods and to the new compounds of the present invention.

With respect to R¹, compounds of formula I wherein R¹ is methyl, ethyl, isopropyl or N(CH₃)₂ are preferred with isopropyl being most preferred.

With respect to R², compounds of formula I wherein R² is hydrogen, methyl or ethyl are preferred, with hydrogen or methyl being most preferred.

With respect to R³, compound of formula I wherein R³ is hydrogen, methyl or ethyl are preferred, with hydrogen or methyl being most preferred.

In addition, when R² is methyl, it is most preferred that R³ is hydrogen, and when R² is hydrogen, it is most preferred that R³ is methyl.

With respect to R^{4a} and R^{4b}, compounds of formula I wherein R^{4a} and R^{4b} are each independently hydrogen, methyl, ethyl, methoxy, ethoxy, Br, Cl or F are preferred, with hydrogen, methyl, methoxy and F being most preferred and hydrogen being most especially preferred.

With respect to n, compounds of formula I wherein n is an integer 1, 2, 3, or 4 are preferred, with 3 or 4 being most preferred.

With respect to R⁶ and R⁷ compounds of formula I wherein R⁶ and R⁷ are (1-4C)alkyl are preferred, with methyl being most preferred.

With respect to R⁹ compounds of formula I wherein R⁹ is (1-6C)alkyl is preferred, with methyl, ethyl, propyl, and isopropyl being most preferred, and methyl and isopropyl being most especially preferred.

With respect to R¹⁰ and R¹¹ compounds of formula I wherein R¹⁰ and R¹¹ are (1-4C)alkyl are preferred with methyl being most preferred.

We claim:

1. A compound of the formula: 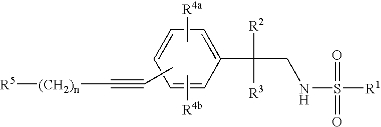
wherein
R¹ represents (1-6C)alkyl, (2-6C)alkenyl, or NR⁶R⁷;
R² and R³ each independently represent hydrogen, (1-4C)alkyl, F, or —OR⁸;
R^{4a} and R^{4b} each independently represent hydrogen, (1-4C) alkyl, (1-4C)alkoxy, I, Br, Cl, or F;
R⁵ represents hydrogen, (1-4C)alkyl, CF₃, OH, OCOCF₃, NH₂, NHCONR¹⁰R¹¹, NHCOR⁹, or NHSO₂R⁹;
n is an integer 1, 2, 3, 4, 5; or 6;
R⁶ and R⁷ each independently represent hydrogen or (1-4C)alkyl;
R⁸ represents hydrogen or (1-4C)alkyl;
R⁹ represents (1-6C)alkyl or NR⁶R⁷; and
R¹⁰ and R¹¹ each independently represent hydrogen or (1-4C)alkyl;
or a pharmaceutically acceptable salt thereof.

2. A compound according to claim 1 wherein R¹ is (1-4C)alkyl.

3. A compound according to claim 2 wherein R¹ is isopropyl.

4. A compound according to claim 3 wherein R² is (1-4C)alkyl and R³ is hydrogen.

5. A compound according to claim 3 wherein R² is methyl and R⁵ is hydrogen.

6. A compound according to claim 5 wherein R^{4a} and R^{4b} are each independently hydrogen, F, methyl, or methoxy.

7. A compound according to claim 6 wherein R⁵ is OH.

8. A compound according to claim 6 wherein R⁵ is NH₂.

9. A compound according to claim 6 wherein R⁵ is NHCOR⁹.

10. A compound according to claim 6 wherein R⁵ is NHSO₂R⁹.

11. A compound according to claim 6 wherein R⁵ is NHCONR¹⁰R¹¹.

12. A compound selected from the group consisting of;
{2-[4-(3-hydroxyprop-1-ynyl)phenyl]propyl}[(methylethyl)sulfonyl]amine;
{2-[4-(4-hydroxybut-1-ynyl)phenyl]propyl}[(methylethyl)sulfonyl]amine;
{2-[4-(5-hydroxypent-1-ynyl)phenyl]propyl}[(methylethyl)sulfonyl]amine;
{2-[4-(6-hydroxyhex-1-ynyl)phenyl]propyl}[(methylethyl)sulfonyl]amine;
{2-[4-(5-aminopent-1-ynyl)phenyl]propyl}[(methylethyl)sulfonyl]amine;
{2-[4-(6-aminohex-1-ynyl)phenyl]propyl}[(methylethyl)sulfonyl]amine;
N-{6-[4-(1-methyl-2-{[(methylethyl)sulfonyl]amino}ethyl)phenyl]hex-5-ynyl}acetamide;
N-{6-[4-(1-methyl-2-{[(methylethyl)sulfonyl]amino}ethyl)phenyl]hex-5-ynyl} methyl sulfonamide;
(dimethylamino)-N-{6-[4-(1-methyl-2-{[(methylethyl)sulfonyl]amino}ethyl)phenyl]hex-5-ynyl}carboxamide;
2-methyl-N-{6-[4-(1-methyl-2-{[(methylethyl)sulfonyl]amino} ethyl)phenyl]hex-5-ynyl}propanamide;
{2-fluoro-2-[4-(6-hydroxyhex-1-ynyl)phenyl]propyl}[(methylethyl)sulfonyl] amine;
2-[4-(6-amino-hex-1-ynyl)-phenyl]-2-fluoro-propyl]-2-propanesulfonamide;
2-fluoro-2-[4-(6-methanesulfonylamino-hex-1-ynyl)-phenyl]propyl-2-propanesulfonamide;
(+)-{2-[4-(6-hydroxyhex-1-ynyl)phenyl]propyl}[(methylethyl)sulfonyl]amine;
(+)-N-{6-[4-(1-methyl-2-{[(methylethyl)sulfonyl]amino}ethyl)phenyl]hex-5-ynyl}trifluoroacetate;
N-{6-[4-(1 methyl-2-{[methylethyl)sulfonyl]amino}ethyl)phenyl]pent-4-ynyl}acetamide;
N-{6-[4-(1 methyl-2-{[methylethyl)sulfonyl]amino}ethyl)phenyl]pent-4-ynyl}acetamide;
N-{6-[-(1-methyl-2-{methylethyl)sulfonyl]amino}ethyl)phenyl]pent-4-ynyl}methyl sulfonamide;
N-{6-[4-(1-methyl-2-methylethyl)sulfonyl]amino}ethyl)phenylpent-4-ynyl}tert butyl carbamate;
2-methyl-N-{6-[4-(methyl-2-{[(methylethyl)sulfonyl]amino}ethyl)phenyl]pent-4ynyl}propanamide;
(dimethylamino-N-{6-[4-(1-methyl-2-{[(methylethyl)sulfonyl]amino} ethyl)phenyl]pent-4-ynyl} carboxamide;
2-methyl-N-{6-[4-(1-methylL-2-{[(methyethyl)sulfonyl]amino} ethyl)phenyl]pent-4-ynyl]ethyl sulfonamide;
{2-fluoro-2-[4-(5-hydroxypent-1-ynyl)phenyl]propyl}[(methylethyl)sulfonyl]amine;
{2-[4-(5-aminopent-1-ynyl)phenyl]propyl}[methylethyl)sulfonyl] amine hydrochloride; and the pharmaceutically acceptable salts thereof.

13. A pharmaceutical composition, which comprises a compound as claimed in claim 1 and a pharmaceutically acceptable diluent or carrier.

14. A method of potentiating glutamate receptor function in a patient, which comprises administering to said patient an effective amount of a compound of formula: 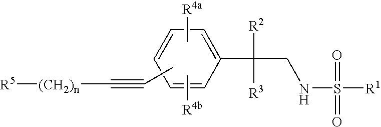
wherein
R¹ represents (1-6C)alkyl, (2-6C)alkenyl, or NR⁶R⁷;
R² R³ each independently represent hydrogen, (1-4C)alkyl, F, or —OR⁸;
R^{4a} and R^{4b} each independently represent hydrogen, (1-4C) alkyl, (1-4C)alkoxy, I, Br, Cl, or F;
R⁵ represents hydrogen, (1-4C)alkyl, CF₃, OH, OCOCF₃, NH₂, NHCONR¹⁰R¹¹, NHCOR⁹, or NHSO₂R⁹;
n is an integer 1, 2, 3, 4, 5; or 6;
R⁶ and R⁷ each independently represent hydrogen or (1-4C)alkyl;
R⁸ represents hydrogen or (1-4C)alkyl;
R⁹ represents (1-6C)alkyl or NR⁶R⁷; and
R¹⁰ and R¹¹ each independently represent hydrogen or (1-4C)alkyl;
or a pharmaceutically acceptable salt thereof.

15. A method of treating a cognitive disorder; Alzheimer's disease, a neuro-degenerative disorder; age-related dementia; age-induced memory impairment; movement disorder; reversal of a drug-induced state; depression; attention deficit disorder; attention deficit hyperactivity disorder; psychosis; cognitive deficits associated with psychosis; drug-induced psychosis, or stroke in a patient, which comprises administering to a patient an effective amount of a compound of formula: 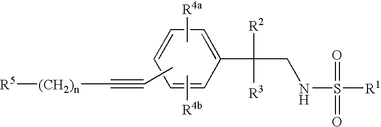
wherein
R¹ represents (1-6C)alkyl, (2-6C)alkenyl, or NR⁶R⁷;
R² and R³ each independently represent hydrogen, (1-4C)alkyl, F, or —OR⁸;
R^{4a} and R^{4b} each independently represent hydrogen, (1-4C) alkyl, (1-4C)alkoxy, I, Br, Cl, or F;
R⁵ represents hydrogen, (1-4C)alkyl, CF₃, OH, OCOCF₃, NH₂, NHCONR¹⁰R¹¹, NHCOR⁹, or NHSO₂R⁹;
n is an integer 1, 2, 3, 4, 5; or 6;
R⁶ and R⁷ each independently represent hydrogen or (1-4C)alkyl;
R⁸ represents hydrogen or (1-4C)alkyl;
R⁹ represents (1-6C)alkyl or NR⁶R⁷; and
R¹⁰ and R¹¹ each independently represent hydrogen or (1-4C)alkyl;
or a pharmaceutically acceptable salt thereof.
